(19) United States
(12) Patent Application Publication
(10) Pub. No.: US 2007/0184109 A1
(43) Pub. Date: Aug. 9, 2007

(54) Compositions comprising triptans and nsaids

(76) Inventors: Alison Green Floyd, Durham, NC (US); Cecilia Carpenter Navy, Durham, NC (US); Gary W. Goodson, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/557,912

(22) PCT Filed: Jun. 2, 2004
(86) PCT No.: PCT/US04/17257

§ 371 Date: Dec. 12, 2006

Related U.S. Application Data
(60) Provisional application No. 60/476,484, filed on Jun. 6, 2003.
(60) Provisional application No. 60/487,413, filed on Jul. 15, 2003.
(60) Provisional application No. 60/490,385, filed on Jul. 25, 2003.

(51) Int. Cl. A61K 31/403; A61K 31/445

(52) U.S. Cl. 424/465; 424/464; 514/319; 514/415

(57) ABSTRACT

A pharmaceutical composition comprising 5HT₁ receptor agonist or a pharmaceutically acceptable derivative thereof in combination with an NSAID or pharmaceutically acceptable derivative thereof wherein the 5HT₁ receptor agonist and NSAID are located in discrete zones with respect to each other wherein each zone comprises the active ingredient and optionally a carrier. A preferred composition comprises sumatriptan succinate and naproxen sodium.

### FIELD OF THE INVENTION

The present invention relates to pharmaceutical compositions comprising more than one active ingredient for oral administration comprising a 5HT₁ receptor agonist in combination with an NSAID (non-steroidal anti-inflamatory drug) as active ingredients, in particular a composition in solid-dosage form that is intended to be swallowed.

### BACKGROUND TO THE INVENTION

The compound 5-hydroxytryptamine (5HT or 5-HT), also known as serotonin or enteramine is a known vasoactive agent and endogenous neurotransmitter acting on receptors both within and outside the central nervous system and on blood vessels. Drugs acting on these receptors may be agonists or antagonists. These receptors have been further classified into several receptor sub classes, some of which themselves also contain subclasses.

5HT₁ receptor agonists are useful in a variety of conditions, notably the treatment of conditions associated with cephalic pain such as migraine, cluster headache, chronic paroxysmal hemicrania, headache associated with vascular disorders, headache associated with substances or their withdrawal, rebound headache and tension headache. 5HT₁ receptor agonists are well known in the art and the term is to be broadly understood to include 5HT₁, receptor agonists of all types including, but not limited to, 5HT_{1F}-like receptor agonists, 5HT_{1B} receptor agonists, 5HT_{1D} receptor agonists and 5HT_{1F} receptor agonists. Particular reference is made to the compounds sumatriptan (described for example in GB Pat. No. 2162522, incorporated herein by reference), naratriptan, rizatriptan, zolmitriptan frovatriptan, eletriptan, almotriptan, avitriptan, donitriptan, alniditan, ALX-0646, LY334370, U109221, IS159 and PNY142633. Especial reference is made to the compound sumatriptan.

An extensive worldwide clinical trials program has demonstrated the efficacy and tolerability of sumatriptan (marketed in subcutaneous, oral, intranasal and rectal formulations) as an acute treatment for migraine.

While 5HT₁ agonists are useful in the treatment of migraine, it has been found that in some patients that after an initial therapeutic effect, migraine symptoms are seen again within about 1-24 hours after the initial relief. That is, after a dosage of the therapeutic agent has been administered to a subject in an amount to effectively treat a migraine, and migraine palliation has been observed, migraine symptoms occur again from as soon as about 1-8 hours after first relief to about 12-24 hours later. It will be appreciated that individual migraineurs display individualized symptoms and timing for this phenomenon as will treatment with particular therapeutic agents.

While not being bound by any particular theory, sumatriptan and other 5-HT agents, are thought to exert their beneficial effect In migraineurs by either reducing the release of pro-inflammatory mediators around certain nerves and blood vessels or by vasoconstriction of selected blood vessels in the head or both. However, they are thought to be devoid of analgesic activity and it is believed that their pharmacologic actions are dependent upon reaching and/or maintaining certain blood concentrations and that these concentrations are relatively short-lived. Relapse within the first 24 hours is well documented and occurs in up to 40-50% of patients who initially obtain relief but the cause is unknown

The headache, which occurs under the circumstances described above, has been variously and interchangeably termed a “rebound”, “relapse”, “recurrent” or “secondary” headache. The terms not withstanding, it is presently unknown as to whether this later headache is a continuation of the physiological chain of events that caused original headache, or a new headache due to other or repeated but unrelated underlying pathology. It is also possible that the follow on headache is a response to therapeutic agents which initially were successful in treating the initial migraine symptoms. The terms “rebound”, “relapse”, “recurrent” and “secondary” (as defined below) are considered synonymous as used herein without interferring a mechanism or cause of the headache described above.

In some forms of migraine, certain patients have found total or partial relief with the use of analgesics such as acetaminophen and phenacetin and other non-steriodal non-opiate analgesics not generally classified as anti-inflammatory. While, these agents, when taken alone, are rarely effective In providing complete and rapid relief of all the symptoms of migraine, especially when the symptoms of the attack already include nausea and vomiting, in combination therapy of the present invention their effectiveness is surprisingly increased.

NSAIDs are well known in the art and particular reference is made to diclofenac, nabumetone, naproxen, ketorolac, ibuprofen, flurbiprofen, ketoprofen, oxaprozin, etodolac, Indomethacin, mefanamic acid, tolfenamic acid and COX-2 selective Inhibitors such as celecoxib, rofecoxib (VIOXX), valdecoxib, parecoxib, 4-(4-cyclohexyl-2-methyl-5-oxazolyl)-2-fluorobenzenesulfonamide (JTE-522), MK633 (etoricoxib), nimesulide, flosulide, DFP, 2-(4-ethoxy-phenyl)-3-(4-methanesulfonyl-phenyl)-pyrazolo[1,5-b]pyridazine, meloxicam, RS57067, piroxicam, NS398, L-745,337 and COX-189.

NSAIDs such as naproxen sodium are thought to relieve migraine pain through their known analgesic action, but may also relieve symptoms by reducing the neurogenic and vascular inflammation secondary to their known anti-inflammatory actions or by other mechanisms such as, but not limited to, platelet inhibition or inhibition of prostaglandin synthesis. In addition, naproxen and naproxen sodium have half-lives on the order of 12-15 hours and produce a long-lasting effect.

U.S. Pat. No. 6,060,499 discloses a method of treating migraine in a human comprising co-timely administering a therapeutically effective amount of a 5HT agonist coordinated with a therapeutically effective amount of an analgesic, particularly a long acting NSAID. Such administration is contemplated in separate formulations or combined in unit dosage form. U.S. Pat. No. 6,060,449 states that without being bound by theory it is believed that combining a 5HT agonist with a long acting NSAID an enhanced initial therapeutic effect may be achieved together with a lower incidence of relapse headaches.

While not being bound by any particular theory, it Is believed that the “relapse” headache often associated with 5-HT agonises is due to the original beneficial effect of the 5-HT agonises wearing off because of their short duration of action while a) the underlying trigger for the original migraine episode is still present and/or b) while the causative agent for the pain and other symptoms, presumably the vascular and/or neurogenic inflammation, still exists.

In this context, the addition of a NSAID to a 5-HT agonist extends the period of effective anti-migraine action and prevents the relapse headache for occurring (or “rebound moderates”), whatever is its cause.

Oral administration constitutes a preferred route for administration of pharmaceuticals since this route Is particularly convenient and acceptable for patients. For combination therapy, administration of one formulation comprising two or more active ingredients may also be preferred. An important consideration in the preparation of formulations containing more than one active ingredient is the stability and efficacy of the ingredients given that the mutual interaction of the agents themselves or the agents with excipients may lead to instability of one or all of the active ingredients or alteration of the efficacy of one or all of the active ingredients.

The present inventors have surprisingly found that when granular preparations of sumatriptan and naproxen sodium were admixed and formulated into one tablet, for example by direct compression, the resultant formulation had an unacceptable dissolution time. For instance, the dissolution of both components was slower than expected leading to slower absorption. This is possibly because the naproxen sodium disables the dissolution of the sumatriptan containing granules.

Surprisingly, when the active ingredients were separated from each other into separate discrete zones with respect to each other and formulated into a solid oral dosage form, the resultant formulation exhibited a satisfactory rate of dissolution of the sumatriptan and the naproxen sodium.

### SUMMARY OF THE INVENTION

Accordingly the invention provides a pharmaceutical composition comprising a 5HT₁ receptor agonist or a pharmaceutically acceptable derivative thereof in combination with an NSAID or pharmaceutically acceptable derivative thereof wherein the 5HT₁ receptor agonist and NSAID are located in discrete zones with respect to each other wherein each zone comprises the active ingredient and optionally a carrier.

### DESCRIPTION OF THE DRAWINGS

FIG. 1 Dissolution Profile of Naproxen Sodium from Anaprox® 550 mg Tablets.

FIG. 2 Dissolution Profiles for Sumatriptan Succinate using FDT Granulation and Naproxen Sodium Direct Compression.

FIG. 3 Dissolution Profiles for Bilayer Tablets Containing Sumatriptan Succinate FDT Granulation and Naproxen Sodium Direct Compression.

FIG. 4 Dissolution Profile for Bilayer Tablet Containing Sumatriptan Succinate FDT Granulation and Naproxen Sodium Granulation.

### DETAILED DESCRIPTION OF THE INVENTION

Suitable 5HT₁ receptor agonists for use according to the invention include sumatriptan, naratriptan, zolmitriptan, eletriptan, rizatriptan, frovatriptan, almotriptan, avitriptan, donitriptan, alniditan, ALX-0646, LY334370, U1092291, IS159 (that is, 3-(2-aminoethyl)-5-[acetamidyl-3-(4-hydroxyphenyl)-propionamidyl-acetamidyl-oxy]-indole) and PNY142633 (that is, (s)-3,4-dihydro-1-[2-[4-[4-aminocarbonyl)phenyl]-1-piperazinyl]ethyl]-N-methyl-1H-2-benzopyran-6-carboximide). Naratriptan and sumatriptan are preferred, with sumatriptan being particularly preferred. A preferred form of sumatriptan is the succinate salt, particularly the 1:1 succinate. The 5HT₁ receptor agonists may be used alone or in combination with each other.

Suitable NSAIDS include diclofenac, nabumetone, naproxen, ketorolac, ibuprofen, flurbiprofen, ketoprofen, oxaprozin, etodolac, indomethacin, mefanamic acid, tolfenamic acid and COX-2 selective inhibitors such as celecoxib, rofecoxib (VIOXX), valdecoxib, parecoxib, 4-(4-cyclohexyl-2-methyl-5-oxazolyl)-2-fluorobenzenesulfonamide (JTE-522), MK633 (etoricoxib), nimesulide, flosulide, DFP, 2-(4-ethoxy-phenyl)-3-(4-methanesulfonyl-phenyl)-pyrazolo[1,5-b]pyridazine, meloxicam, RS57067, piroxicam, NS398, L-745,337 and COX-189. Preferred are COX-189 and naproxen. Particularly preferred is naproxen, most preferably in the form of a naproxen sodium.

By “pharmaceutically acceptable derivative” is meant any pharmaceutically acceptable salt, solvate, ester or amide or salt or solvate of such ester or amide of the active ingredient or any other compound which, on administration to the recipient is capable of providing (directly or indirectly) the active ingredient or an active metabolic or residue thereof.

Suitable pharmaceutically acceptable salts according to the invention include acid addition salts formed with Inorganic acids such as hydrochlorides, hydrobromides, phosphates and sulfates and with organic acids, for example tarbrates, maleaves, fumerates, succinates and sulfonates.

In one aspect of the Invention, the discrete zones may be provided by the addition of excipients. Therefore the invention provides a pharmaceutical composition, comprising a 5HT₁ receptor agonist or a pharmaceutically acceptable derivative thereof and a pharmaceutically acceptable carrier in combination with an NSAID, wherein the 5HT₁ receptor agonist and NSAID are each dispersed within its own pharmaceutically acceptable carrier in the pharmaceutical composition.

Suitably, the carrier for the 5HT₁ receptor agonist, is different in composition to that of the carrier for the NSAID.

In one embodiment of the invention the 5HT₁ receptor agonist and its carrier are substantially in admixture with the NSAID and its carrier.

Suitably the 5HT₁ receptor agonist and its carrier are substantially in homogenous admixture with the NSAID and its carrier.

Suitably, the pre-formed 5HT₁ receptor agonist/carrier mixture is admixed with the pre-formed NSAID/carrier mixture on preparation of the composition of the invention. For example the 5HT₁ receptor agonist/carrier mixture is admixed with the NSAID/carrier mixture in a capsule form.

More preferably, the 5HT₁ receptor agonist/carrier mixture is compacted with the NSAID/carrier mixture in the composition, suitably to form a tablet. For example, the admixture of the 5HT₁ receptor agonist/carrier mixture with the NSAID/carrier mixture is compacted to form a tablet.

A suitable carrier for the 5HT₁ receptor agonist comprises one or more components selected from: a binding agent, a filler, a lubricant, and effervescent couple, a wicking agent, a glidant, a disintegrant and a wetting agent.

Suitable carriers for the NSAID comprises one or more components selected from: a binding agent, a filler, a lubricant, an effervescent couple, a wicking agent, a glidant, a disintegrant and a wetting agent.

In a more preferred aspect of the invention, a layer provides a suitable zone, generally a compressed layer, of the active agent. Thus, the formulation may comprise layers, generally shaped layers of the active agents.

A suitable formulation is a tablet formulation. Thus one particular formulation is a multilayer tablet wherein the active agents are in separate layers. One particular formulation comprises a compressed form, for example a tablet, of one active agent formulated with a powdered form of the other active agent. Alternatively, a tablet can be prepared by overcompression such that it would include a core of one active ingredient surrounded by an over coat of another active ingredient.

More preferably, the tablets containing active agents in discrete zones with respect to each other may be multilayer tablets. For example they may be bilayer tablets, where a layer of the form of one active agent is compressed, the form of the other active agent then added and compressed onto the layer of the first active agent. They may also be trilayer tablets prepared in an analogous manner. The active ingredients may be formulated as granules if appropriate.

The discrete zones may be separated by a barrier layer, preferably an inert barrier layer. The barrier layer conveniently comprises a filler, such as lactose, and a lubricant, such as magnesium stearate

As indicated, such compositions may conveniently be produced as tablets or capsules. Tablets and capsules may be produced by admixture of granular forms of the active agents followed by compression.

Granules of each active agent may be obtained by combination of the active agent with appropriate excipients, for example hydroxypropyl methyl cellulose, microcrystalline cellulose, sodium starch glycollate, lactose, and magnesium stearate, followed by granulation using conventional techniques.

Where granules of either active agent are produced, preferably high shear granulation techniques are employed. In a preferred embodiment, granulation of both active agents is carried out.

Capsules may be produced by admixture of pelleted forms or granular forms of the active agents followed by encapsulation.

Pellets of each active agent may be obtained by combination of the active agent with appropriate excipients, for example microcrystalline cellulose and lactose, followed by pellet formation using conventional techniques. Granules are prepared as described herein. The production of tablets and capsules may be undertaken using techniques that are well known in the art.

In the context of the present invention, by “for oral administration” is meant that the pharmaceutical composition is in a solid-dosage form that is intended to be swallowed whole, and is not primarily intended for dissolution or suspension in water prior to administration; however, the composition of the present invention may in certain embodiments meet the requirements of a dispersible tablet in terms of fineness of dispersion and rate of dispersion as defined by the European Pharmacopoeia and/or British Pharmacopoeia. Such dosage forms may take the form of tablets and capsules and may be prepared according to conventional techniques well known in the art of pharmacy for the manufacture of solid-dosage forms. Preferably, the composition of the present invention is in the form of a ‘swallow’ tablet. For the avoidance of doubt, a ‘swallow’ tablet is a tablet which is intended to be swallowed whole (generally with a small amount of liquid, e.g. water); it is not one which is primarily intended for dissolution or suspension In water prior to administration (such as, for example, a tablet as described in WO92/11003 and which also contains a substantial quantity of both components of an effervescent couple), and also it is not one which is primarily intended for dissolution ‘in the mouth’ (in an ‘oral melt’ form).

WO 92/15295 (Glaxo Group Ltd.) incorporated herein by reference discloses known solid-dosage form pharmaceutical compositions, in particular film coated tablets useful in the treatment of conditions associated with cephalic pain. These formulations may form the 5HT₁ receptor agonist containing zone.

Unfortunately there may be certain disadvantages when treating conditions associated with cephalic pain. For example such conditions, particularly migraine, are associated with nausea, vomiting and gastrointestinal dysfunction in the form of reduced gastric mobility and delayed gastric emptying which potentially lead to delay and/or impairment of drug absorption.

It would thus be desirable to have the active Ingredients, particularly the 5HT₁ receptor agonist formulated to ensure rapid dissolution and absorption. Thus in a preferred aspect of the invention there Is provided a pharmaceutical composition comprising a 5HT₁ receptor agonist or a pharmaceutically acceptable derivative thereof in combination with an NSAID or pharmaceutically acceptable derivative thereof wherein the 5HT₁ receptor agonist and NSAID are located in discrete zones with respect to each other wherein each zone comprises the active ingredient and optionally a carrier and further wherein at least the zone comprising the 5HT₁ receptor agonist is formulated to ensure rapid absorption.

By “rapid absorption” applied to 5HT₁ receptor agonist is meant that greater than about 70%, preferably greater than about 80%, more preferably greater than about 90% of the active ingredient is dissolved in simulated gastric fluid (SGF) within five minutes in USPII apparatus at the discriminating paddle speed of 10 rpm.

In a preferred aspect the formulation is a tablet comprising 2 or more layers wherein one layer comprising a 5HT₁ receptor agonist or pharmaceutically acceptable derivative thereof is formulated to ensure rapid absorption and another layer comprises an NSAID or pharmaceutically acceptable derivative thereof and optionally a carrier. Most preferably the tablet is a bilayer tablet comprising a layer comprising a 5HT₁ receptor agonist or pharmaceutically acceptable derivative thereof which is formulated to ensure rapid absorption and a layer comprising an NSAID or pharmaceutically acceptable derivative thereof and optionally a carrier. Thus in a preferred aspect, in a 5HT₁ receptor agonist layer formulated for rapid absorption, the 5HT₁ receptor agonist or pharmaceutically acceptable derivative thereof Is formulated together with an effervescent couple in combination with a disintegrant, a filler (preferably an insoluble filler), and a wicking agent. This formulation has been shown to have a faster onset of action and/or higher efficacy rate for a patient suffering from cephalic pain.

A layer of the pharmaceutical formulation comprising the NSAID or pharmaceutically acceptable derivative thereof may be formulated according to well known NSAID formulations. These will be apparent to a person skilled in the art. For example, compositions of several different formulations of naproxen sodium tablets are shown in the table below.

Composition (mg/tablet)

| Ingredient | 1 | 2 | 3 | 4 | 5 | 6 | 7 | 8 |
|---|---|---|---|---|---|---|---|---|
| Naproxen Sodium | 550 | 550 | 550 | 550 | 550 | 550 | 550 | 550 |
| Avicel PH 101 | 164 | 164 | 164 | 126 | 126 | 164 | 164 | 164 |
| PVP K-30, USP | — | 20 | — | 20 | — | 20 | 20 | 20 |
| PVP K-90, USP | — | — | 20 | — | 20 | — | — | — |
| Purified Water, | — | 110 | 110 | 110 | 110 | 125 | 150 | 175 |
| USP | | | | | | | | |
| Ac-Di-Sol | — | — | — | 38 | 38 | — | — | — |
| Talc, USP | 8 | 8 | 8 | 8 | 8 | 8 | 8 | 8 |
| Magnesium | 8 | 8 | 8 | 8 | 8 | 8 | 8 | 8 |
| Stearate | | | | | | | | |
| Total Weight | 730 | 750 | 750 | 750 | 750 | 750 | 750 | 750 |

Bansal, P., Haribhakti, K., Subramanian, V., Plakogiannis, F., “Effect of Formulation and Process Variables on the Dissolution Profile of Naproxen Sodium from Tablets”, Drug Development and Industrial Pharmacy, 20(13), 2151-2156, 1994.

In a further aspect of the invention, the NSAID layer also may be formulated for rapid absorption, e.g. by the inclusion of an effervescent couple.

By “urapid absorption” applied to the NSAID is meant that greater than about 25%, preferably greater than about 50%, more preferably greater than about 75% of the active ingredient is dissolved in simulated intestinal fluid within five minutes in USPII apparatus at the discriminating paddle speed of 30 rpm.

Thus in a further aspect of the invention, in an NSAID layer, the NSAID or pharmaceutically acceptable derivative thereof is formulated together with an effervescent couple in combination with a disintegrant, an insoluble filler and a wicking agent.

Accordingly, in a preferred embodiment the present invention provides in a 5HT₁ receptor agonist layer formulated for rapid absorption, the 5HT₁ receptor agonist or a pharmaceutically acceptable derivative thereof, formulated together with the base component of an effervescent couple, a disintegrant and an insoluble filler, wherein the base component comprises from about 5 to about 50% by weight, the disintegrant comprises from about 0.5 to about 10% by weight, and the insoluble filler comprises from about 20 to about 99% by weight, said insoluble filler including a wicking agent which comprises from about 1 to about 99% by weight, based on the dry weight of the layer of the dosage form, wherein greater than about 70%, preferably about 80%, more preferably about 90% of the active ingredient is dissolved in simulated gastric fluid (SGF) within five minutes in USPII apparatus at the discriminating paddle speed of 10 rpm.

When the NSAID is also formulated for rapid absorption, the invention provides an NSAID layer formulated for rapid absorption comprising the NSAID or a pharmaceutically acceptable derivative thereof, formulated together with the base component of an effervescent couple, a disintegrant and an insoluble filler, wherein the base component comprises from about 5 to about 50% by weight, the disintegrant comprises from about 0.5 to about 20% by weight, and the insoluble filler comprises from about 30 to about 80% by weight, said insoluble filler including a wicking agent which comprises from about 1 to about 60% by weight, based on the dry weight of the layer of the dosage form, wherein greater than about 25%, preferably about 50%, more preferably about 75% of the active ingredient is dissolved in simulated intestinal fluid within five minutes in USPII apparatus at the discriminating paddle speed of 30 rpm.

Suitably, in a 5HT₁ receptor agonist layer formulated for rapid absorption, the 5HT₁ receptor agonist comprises from about 0.001 to about 55% by weight, preferably about 0.01 to about 45%, more preferably about 0.01 to about 40%, especially about 1 to about 35%, more especially about 20 to about 35% based on the dry weight of the layer.

Suitably, in a NSAID layer, the NSAID comprises from about 1 to about 90% by weight, preferably about 2 to about 90%, more preferably about 5 to about 85%, especially about 10 to about 75%, more especially about 60 to about 75% based on the dry weight of the layer.

In one embodiment of the present invention, layers formulated for rapid absorption, the pharmaceutical composition further comprises the acid component of an effervescent couple. An effervescent couple consists essentially of an acid component and a base component, which components react in the presence of water to form a gas.

In the 5HT₁ receptor agonist layer formulated for rapid absorption, the acid component may comprise, for example, the 5HT₁ receptor agonist or a pharmaceutically acceptable derivative thereof itself (where it has an acidic character or can provide a component with acidic character in an aqueous environment), or an aliphatic carboxylic acid or a salt thereof, such as citric or tartaric acid and salts thereof. Alternatively, the acid component may be provided by acid in the NSAID layer or the stomach acid rather than being part of the 5HT₁ receptor agonist zone of the pharmaceutical composition. Preferably, the acid component is the 5HT₁ receptor agonist or a pharmaceutically acceptable derivative thereof (such as sumatriptan or naratriptan, especially in the form of salts thereof, for example the succinate salt, such as sumatriptan succinate (1:1)). The acid components may be used alone or in combination with each other. Suitably, the acid component comprises the 5HT₁ receptor agonist or a pharmaceutically acceptable derivative thereof, together with an aliphatic carboxylic acid or a salt thereof, such as citric or tartaric acid and salts thereof. Suitably, the acid component (including the 5HT₁ receptor agonist or pharmaceutically acceptable derivative thereof when it is functioning as an acid component) comprises up to about 55% by weight, preferably from about 5 to about 50%, more preferably about 10 to about 45%, especially about 15 to about 40%, more especially about 20 to about 35%, based on the dry weight of the layer of the dosage form. The base component may comprise, for example, an alkali metal or alkaline earth metal carbonate or bicarbonate, such as sodium bicarbonate, potassium bicarbonate, magnesium carbonate or calcium carbonate. The base component is preferably sodium bicarbonate. The base components may be used alone or in combination with each other. Suitably, the base component comprises from about 5 to about 50% by weight, preferably about 7 to about 20%, more preferably about 8 to about 15%, especially about 9 to about 12%, based on the dry weight of the layer of the dosage form. Acids may be monoprotic or polyprotic; similarly, bases may be monobasic or polybasic. Calculated in terms of acid/base normalities (N), the ratio of acid component to base component may conveniently be within the range of from about 1:10 to about 10:1, preferably from about 1:5 to about 5:1, more preferably about 1:3 to about 3:1, most preferably about 1:2 to about 2:1.

In the NSAID layer formulated for rapid absorption, the acid component may comprise, for example, the NSAID such as naproxen or a pharmaceutically acceptable derivative thereof itself (where it has an acidic character or can provide a component with acidic character in an aqueous environment), or an aliphatic carboxylic acid or a salt thereof, such as citric or tartaric acid and salts thereof. Alternatively, the acid component may be provided by acid in the 5HT₁ receptor agonist layer or the stomach acid rather than being part of the NSAID layer of the pharmaceutical composition, or as a separate pharmaceutical agent such as citric acid included either in the 5HT₁ receptor agonist layer and/or the NSAID layer. Preferably, the acid component is provided as a separate pharmaceutical agent such as citric acid, The acid components may be used alone or in combination with each other. Suitably, the acid component comprises the 5HT₁ receptor agonist or a pharmaceutically acceptable derivative thereof, together with an aliphatic carboxylic acid or a salt thereof, such as citric or tartaric acid and salts thereof. Suitably, the acid component included as a separate pharmaceutical agent such as citric acid up to about 30% by weight, preferably from about 0.001 to about 20%, more preferably about 0.01 to about 15%, especially about 1 to about 15%, more especially about 3 to about 10%, based on the dry weight of the layer of the dosage form. The base component may comprise, for example, an alkali metal or alkaline earth metal carbonate or bicarbonate, such as sodium bicarbonate, potassium bicarbonate, magnesium carbonate or calcium carbonate. The base component is preferably sodium bicarbonate. The base components may be used alone or in combination with each other. Suitably, the base component comprises from about 5 to about 50% by weight, preferably about 7 to about 20%, more preferably about 8 to about 15%, especially about 9 to about 12%, based on the dry weight of the layer of the dosage form. Acids may be monoprotic or polyprotic; similarly, bases may be monobasic or polybasic. Calculated in terms of acid/base normalities (N), the ratio of acid component to base component may conveniently be within the range of from about 1:10 to about 10:1, preferably from about 1:5 to about 5:1, more preferably about 1:3 to about 3:1, most preferably about 1:2 to about 2:1.

Disintegrants, when used in the compositions of the present invention, swell when they come into contact with water. Suitable disintegrants will be well known to the person skilled in the art and a non-limiting list of examples includes croscarmellose sodium, sodium starch glycollate, cross-linked polyvinylpyrrolidone, povidone, starch (e.g. maize starch, pregelatinised starch), low substituted hydroxypropylcellulose, alginic acid, sodium alginate, tribasic calcium phosphate, calcium sulfate, calcium carboxymethylcellulose, microcrystalline cellulose, powdered cellulose, colloidal silicon dioxide, docusate sodium, guar gum, hydroxypropyl cellulose, magnesium aluminium silicate, methylcellulose, polacrilin potassium and polyvinyl pyrrolidone. Croscarmellose sodium is preferred. The disintegrants may be used alone or in combination with each other. Suitably, in the 5HT₁ receptor agonist zone the disintegrant comprises from about 0.5 to about 10% by weight, preferably about 2 to about 8%, more preferably about 3 to about 7%, especially about 4 to about 6%, more especially about 5%, based on the dry weight of the layer of the dosage form.

In the NSAID layer the disintegrant comprises from about 0.5 to about 10% by weight, preferably about 1 to about 8%, more preferably about 1.5 to about 6%, especially about 2 to about 4%, more especially about 3%, based on the dry weight of the of the layer dosage form.

Insoluble fillers are inert substances that provide bulk and stability when used in the compositions of the present invention. Some insoluble fillers may also act as wicking agents. Wicking agents, when used in the compositions of the present invention, have a porous nature and draw water into and throughout the solid dosage form. Suitable wicking agents will be well known to the person skilled in the art and a non-limiting list of examples includes microcrystalline cellulose (available as, for example, Avice™), croscarmellose sodium, crospovidone, starch, calcium carboxymethylcellulose, silicified microcrystalline cellulose, magnesium oxide and tragacanth. Microcrystalline cellulose is preferred. The wicking agents may be used alone or in combination with each other. Suitably, the wicking agent comprises from about 1 to about 99% by weight, preferably about 1 to about 80%, more preferably about 5 to about 65%, especially about 12 to about 55%, more especially about 18 to about 50%, based on the dry weight of the layer of the dosage form. Other suitable insoluble fillers include dibasic calcium phosphate dihydrate, anhydrous dibasic calcium phosphate (available as, for example, Emcompress™), tribasic calcium phosphate, calcium carbonate, magnesium carbonate, calcium sulfate, cellulose acetate, powdered cellulose, kaolin, polymethacrylates and talc. Anhydrous dibasic calcium phosphate is preferred. The insoluble fillers may be used alone or in combination with each other. Suitably, in the 5HT₁ receptor agonist layer the insoluble filler, including the wicking agent, comprises from about 30 to about 99% by weight, preferably about 35 to about 80%, more preferably about 40 to about 70%, especially about 45 to 65%, based on the dry weight of the layer of the dosage form. In the NSAID layer the insoluble filler, including the wicking agent, comprises from about 10 to about 99% by weight, preferably about 15 to about 55%, more preferably about 25 to about 45%, especially about 30 to 40%, based on the dry weight of the layer of the dosage form.

Thus, in an embodiment of the present invention, there is provided a solid-dosage form pharmaceutical composition as hereinbefore described wherein the 5HT₁ receptor agonist layer comprises a 5HT₁ receptor agonist or a pharmaceutically acceptable derivative thereof which comprises from about 0.001 to about 55% by weight, preferably about 0.01 to about 45%, more preferably about 0.1 to 40%, especially about 1 to about 35%, more especially about 20 to about 35%, the base component of the effervescent couple comprises from about 5 to about 50% by weight, preferably about 7 to about 20%, more preferably about 8 to about 15%, especially about 9 to about 12%, the disintegrant comprises from about 0.5 to about 10% by weight, preferably about 2 to about 8%, more preferably about 3 to about 7%, especially about 4 to about 6%, more especially about 5%, the insoluble filler, including the wicking agent, comprises from about 35 to about 80% by weight, preferably about 40 to about 70%, more preferably about 45 to about 65%, and the wicking agent comprises from about 1 to about 80% by weight, preferably about 5 to about 65%, more preferably about 12 to about 55%, especially about 18 to about 50%, based on the dry weight of the layer of the dosage form.

Thus, in an embodiment of the present invention, there is provided a solid-dosage form pharmaceutical composition as hereinbefore described wherein the NSAID layer comprises a NSAID or a pharmaceutically acceptable derivative thereof which comprises from about 1 to about 90% by weight, preferably about 2 to about 90%, more preferably about 5 to about 85%, especially about 10 to about 75%, more especially about 60 to about 75%, the base component of the effervescent couple comprises from about 5 to about 50% by weight, preferably about 7 to about 20%, more preferably about 8 to about 15%, especially about 9 to about 12%, the disintegrant comprises from about 0.05 to about 10% by weight, preferably about 1 to about 8%, more preferably about 1.5 to about 6%, especially about 2 to about 4%, more especially about 3%, the insoluble filler, including the wicking agent, comprises from about 10 to about 99% by weight, preferably about 15 to about 55%, more preferably about 25 to about 45%, based on the dry weight of the layer of the dosage form.

Thus, in a particularly preferred embodiment of the present invention, there is provided a solid-dosage form brayer tablet as hereinbefore described wherein the 5HT₁ receptor agonist layer comprises a 5HT₁ receptor agonist or a pharmaceutically acceptable derivative thereof which comprises from about 0.001 to about 55% by weight, preferably about 0.01 to about 45%, more preferably about 0.1 to 40%, especially about 1 to about 35%, more especially about 20 to about 35%, the base component of the effervescent couple comprises from about 5 to about 50% by weight, preferably about 7 to about 20%, more preferably about 8 to about 15%, especially about 9 to about 12%, the disintegrant comprises from about 0.5 to about 10% by weight, preferably about 2 to about 8%, more preferably about 3 to about 7%, especially about 4 to about 6%, more especially about 5%, the insoluble filler, including the wicking agent, comprises from about 35 to about 80% by weight, preferably about 40 to about 70%, more preferably about 45 to about 65%, and the wicking agent comprises from about 1 to about 80% by weight, preferably about 5 to about 65%, more preferably about 12 to about 55%, especially about 18 to about 50%, based on the dry weight of the layer of the dosage form and the NSAID layer comprises a NSAID or a pharmaceutically acceptable derivative thereof which comprises from about 1 to about 90% by weight, preferably about 2 to about 90%, more preferably about 5 to about 85%, especially about 10 to about 75%, more especially about 60 to about 75%, the base component of the effervescent couple comprises from about 5 to about 50% by weight, preferably about 7 to about 20%, more preferably about 8 to about 15%, especially about 9 to about 12%, the disintegrant comprises from about 0.05 to about 10% by weight, preferably about 1 to about 8%, more preferably about 1.5 to about 6%, especially about 2 to about 4%, more especially about 3%, the insoluble filler, including the wicking agent, comprises from about 10 to about 99% by weight, preferably about 15 to about 55%, more preferably about 25 to about 45, based on the dry weight of the layer of the dosage form.

In a further embodiment of the present invention, there is provided a pharmaceutical composition as hereinbefore described wherein the 5HT₁ receptor agonist zone comprises a 5HT₁ receptor agonist or a pharmaceutically acceptable derivative thereof which comprises sumatriptan or naratriptan or a pharmaceutically acceptable derivative thereof, preferably sumatriptan or a pharmaceutically acceptable derivative thereof, more preferably in the form of its succinate (1:1) salt, the base component of the effervescent couple comprises sodium bicarbonate, the disintegrant comprises croscarmellose sodium and the insoluble filler comprises microcrystalline cellulose.

In a further embodiment of the present invention, there is provided a solid-dosage form pharmaceutical composition as hereinbefore described wherein the 5HT₁ receptor agonist zone comprises a 5HT₁ receptor agonist or a pharmaceutically acceptable derivative thereof which comprises sumatriptan or naratriptan or a pharmaceutically acceptable derivative thereof, preferably sumatriptan or a pharmaceutically acceptable derivative thereof, more preferably in the form of its succinate (1:1) salt, the base component of the effervescent couple comprises sodium bicarbonate, the disintegrant comprises croscarmellose sodium and the insoluble filler comprises dibasic calcium phosphate, preferably anhydrous dibasic calcium phosphate.

In a further embodiment of the present Invention, there is provided a solid-dosage form pharmaceutical composition as hereinbefore described wherein the 5HT₁ receptor agonist zone comprises a 5HT₁ receptor agonist or a pharmaceutically acceptable derivative thereof which comprises sumatriptan or a pharmaceutically acceptable derivative thereof, preferably in the form of its succinate (1:1) salt, the base component of the effervescent couple comprises sodium bicarbonate, the disintegrant comprises croscarmellose sodium, and the insoluble filler comprises anhydrous dibasic calcium phosphate or microcrystalline cellulose or a mixture thereof.

The inclusion of one or more insoluble fillers in the compositions of the present invention also provides the composition with improved handling properties during manufacture as compared with a conventional tablet formulation (for example, a formulation as disclosed in WO92/15295).

In addition to the ingredients described above, the pharmaceutical composition of the present invention may further comprise pharmaceutically acceptable carriers and excipients such as binding agents (e.g. pregelatinised starch, polyvinylpyrrolidone, hydroxypropylmethylcellulose) and lubricants (e.g. stearic acid, magnesium stearate, talc, sodium benzoate and hydrogenated vegetable oil).

Many drug substances have an inherently bitter taste. The unpleasant taste that is sometimes associated with oral administration of a composition comprising a 5HT₁ receptor agonist or a pharmaceutically acceptable derivative thereof may be substantially eliminated by the use of a film coat on the solid core. The solid core comprises the 5HT₁ receptor agonist or a pharmaceutically acceptable derivative thereof. Moreover, when used in the compositions of the present invention, the film coat delays the disintegration of the solid dosage form until it reaches the stomach. A film coat can also aid swallowing, can make the solid dosage form aesthetically more pleasing, and generally makes the solid-dosage form less friable.

Accordingly, In one embodiment, the present Invention provides a pharmaceutical composition as hereinbefore described in the form of a tablet which is film-coated.

The film coating may suitably comprise a polymer. Suitable polymers will be well known to the person skilled in the art and a non-limiting list of examples includes cellulose ethers, for example, hydroxypropylmethyl cellulose, hydroxypropyl cellulose or methylcellulose, and copolymers of methacrylic acid and methyl methacrylate. Preferably, the film coating will comprise hydroxypropylmethyl cellulose.

The total film coating solids are generally applied to the solid dosage form, for example the tablet core, in an amount of from about 0.5 to 10% by weight, preferably about 1 to about 4%, more preferably about 2 to about 3%, based on the dry weight of the dosage form. For example, about 8 mg of coat is used for a tablet core weighing about 300 or about 400 mg, and about 4 mg of coat is used for a tablet core weighing about 175 mg.

The film coating may additionally comprise any pharmaceutically acceptable colourants or opacifiers including water soluble dyes, aluminium lakes of water soluble dyes and inorganic pigments such as titanium dioxide and iron oxide.

The film coating may also contain one or more plasticising agents conventionally used in polymeric film coatings, for examples, polyethylene glycol, propylene glycol, dibutyl sebecate, mineral oil, sesame oil, diethyl phthalate and triacetin. Proprietary film coating materials, such as Opaspray and Opadry, obtainable from Colorcon Ltd., UK, may be used.

The taste of oral compositions may also be improved by the use of flavouring and/or sweetening agents. Suitable flavouring agents will be well known to the person skilled in the art and a non-limiting list of examples includes lemon, orange, grapefruit, vanilla, caramel, butterscotch, hazelnut or mint flavouring. Suitable sweetening agents for use in the compositions of the invention will be well known to the person skilled in the art and a non-limiting list of examples includes sucrose, saccharin, cyclamic acid and alkali or alkali earth metal salts thereof, mannitol, acesulfame-K, stevioside, thaumatin and aspartame. The flavouring and/or sweetening agents may be used alone or In combination with each other.

In a further aspect, the present invention provides a solid-dosage form pharmaceutical composition for oral administration as hereinbefore described for use in the treatment of conditions associated with cephalic pain such as cluster headache, chronic paroxysmal hemicrania, headache associated with vascular disorders, headache associated with substances or their withdrawal, rebound headache, tension headache and, in particular, migraine. Suitably the 5HT₁ receptor agonist is sumatriptan or naratriptan or a pharmaceutically acceptable derivative thereof, preferably sumatriptan or a pharmaceutically acceptable derivative thereof, more preferably sumatriptan succinate (1:1) salt. Suitably the NSAID is naproxen or COX-189 or pharmaceutically acceptable salts thereof, more preferably naproxen sodium.

It will be appreciated that reference to treatment is intended to include prophylaxis as well as the alleviation of established symptoms.

According to a further aspect of the present invention, there is provided the use of a solid-dosage form pharmaceutical composition for oral administration as hereinbefore described in the manufacture of a medicament for treating conditions associated with cephalic pain such as cluster headache, chronic paroxysmal hemicrania, headache associated with vascular disorders, headache associated with substances or their withdrawal, rebound headache, tension headache and, in particular, migraine. Suitably, the 5HT₁ receptor agonist or a pharmaceutically acceptable derivative thereof is sumatriptan or naratriptan or a pharmaceutically acceptable derivative thereof, preferably sumatriptan or a pharmaceutically acceptable derivative thereof, more preferably sumatriptan succinate (1:1) salt. Suitably the NSAID is naproxen or COX-189 or pharmaceutically acceptable salts thereof, more preferably naproxen sodium.

A further aspect of the present invention provides a method of treating a mammal, including a human, suffering from or susceptible to conditions associated with cephalic pain such as cluster headache, chronic paroxysmal hemicrania, headache associated with vascular disorders, headache associated with substances or their withdrawal, rebound headache, tension headache and, In particular, migraine, which comprises oral administration of a solid-dosage form pharmaceutical composition as hereinbefore described. Suitably, the 5HT₁ receptor agonist or a pharmaceutically acceptable derivative thereof is sumatriptan or naratriptan or a pharmaceutically acceptable derivative thereof, preferably sumatriptan or a pharmaceutically acceptable derivative thereof, more preferably sumatriptan succinate (1:1) salt. Suitably the NSAID is naproxen or COX-189 or pharmaceutically acceptable salts thereof, more preferably naproxen sodium.

It will be appreciated that the amount of compounds employed as the active ingredient in the solid-dosage form compositions of the invention will depend on the particular compounds used. Furthermore, the precise therapeutic dose employed will depend on the age and condition of the patient and the nature of the condition to be treated and will be at the ultimate discretion of the attendant physician.

When the 5HT₁ receptor agonist is sumatriptan or naratriptan, the amount of compound employed In the compositions of the invention will be in the range of 0.1 mg to 250 mg. The compositions may be administered for example 1 to 4 times per day, preferably once or twice. When the 5HT₁ receptor agonist is sumatriptan, the amount of sumatriptan, preferably in the form of a pharmaceutically acceptable salt, will be in the range of 1 mg to 200 mg, preferably 20 mg to 150 mg, for example 25, 50, 85 or 100 mg, expressed as the weight of free base. When the 5HT₁ receptor agonist is naratriptan, the amount of naratriptan, preferably in the form of a pharmaceutically acceptable salt, will be in the range of 0.1 mg to 25 mg, preferably 1 or 2.5 mg, expressed as the weight of free base. When the NSAID is naproxen or COX-189, the amount of compound employed in the compositions of the invention will be in the range of 25 mg to 1100 mg. When the NSAID is naproxen, the amount of naproxen, preferably in the form of a pharmaceutically acceptable salt, will be in the range of 100 mg to 1100 mg, preferably 250 mg to 800 mg, for example 454.5 mg or 500 mg, expressed as the weight of free acid, or 275, 350, 400, 500 or 550 mg, expressed as the weight of the sodium salt.

In a particularly preferred embodiment the pharmaceutical composition of the invention is a bilayer tablet comprising 85 mg sumatriptan in the form of 119 mg sumatriptan succinate and 550 mg naproxen sodium equivalent to 500 mg naproxen wherein at least the sumatriptan is formulated for rapid absorption.

In a particularly preferred embodiment the pharmaceutical composition of the invention is a bilayer tablet comprising 85 mg sumatriptan in the form of 119 mg sumatriptan succinate and 500 mg naproxen sodium equivalent to 454.5 mg naproxen wherein at least the sumatriptan is formulated for rapid absorption.

Typically In the treatment of cephalic pain, in particular migraine, the composition is administered as a single dose; if a patient experiences rebound pain or recurrance then a subsequent single dose may be administered after a suitable period in accordance with the instructions provided by the attendant physician. Thus, the treatment of cephalic pain, in particular migraine, envisaged in the context of the present invention is essentially by means of acute, single dose, administration of the active ingredient. The compositions may be administered for example 1 to 4 times per day, preferably once or twice.

The invention will now be illustrated by the following Examples which should not be construed as a limitation thereto

### EXAMPLE 1

Sumatriptan Succinate Granulation and Naproxen Sodium Direct Compression Tablets

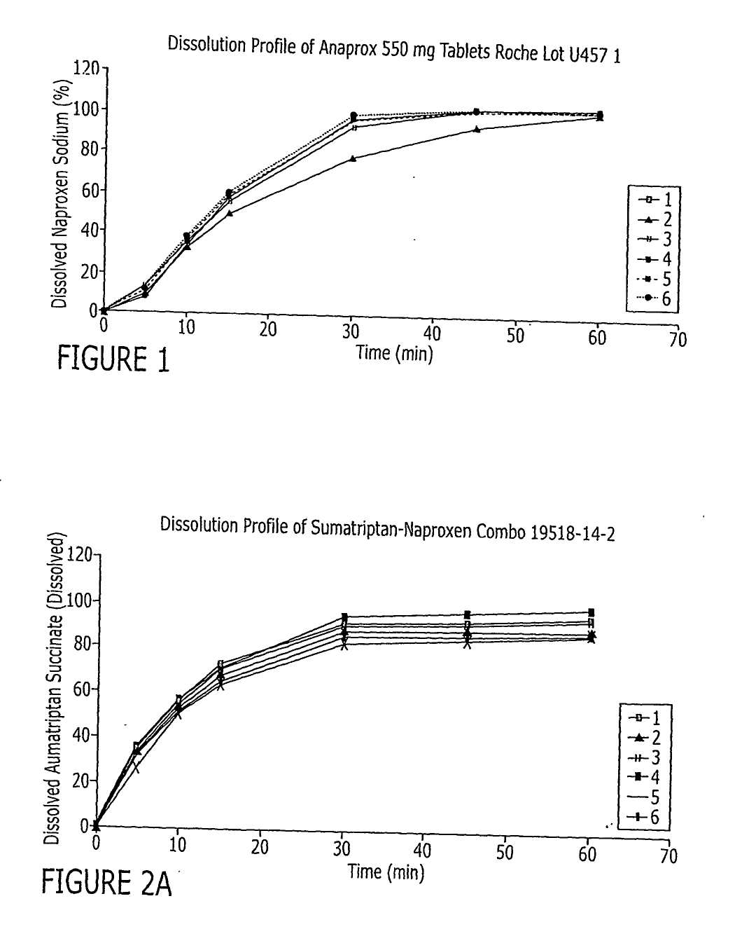

The tablets were prepared by compression on a hydraulic Carver press using conventional techniques. The aim was to prepare a single tablet providing rapid dissolution of 119 mg of sumatriptan succinate (equivalent to 85 mg sumatriptan free base) and appropriate release rate of 550 mg naproxen sodium (equivalent to 500 mg naproxen free acid). A target dissolution profile for sumatriptan succinate was for 95% to be dissolved in 5 minutes at dissolution test conditions previously described. As for naproxen sodium, the target profile was one that was competitive with the gold standard, Anaprox® DS (550 mg naproxen sodium, Roche), which released approximately 57% in 15 minutes, under the same dissolution test conditions. FIG. 1 shows the dissolution profile obtained for naproxen sodium from Anaprox® 550 mg commercial tablets.

The initial step of formulation development was to determine the feasibility of a combined direct compression of the two drug substances. Formulations were prepared with various fillers and binders (Anhydrous Emcompress®, Avicel PH® 102, and Prosolv SMCC™ 90) and select disintegrants, glidants and lubricants, and the available Montrose-sourced sumatriptan succinate and naproxen sodium USP, from Albemarle Corporation. Representative formulations are presented in Table 1.

TABLE 1

| | 1 | | 2 | | 3 | |
|---|---|---|---|---|---|---|
| Ingredients | Qty (mg) | | Qty (mg) | | Qty (mg) | |
| Naproxen | 550 | | 550 | | 550 | |
| Sodium | | | | | | |
| Sumatriptan | 120 | | 120 | | 120 | |
| Succinate | | | | | | |
| Filler/Binders | | | | | | |
| Anhydrous | 327.7 | (29.79%) | — | | — | |
| Emcompress | | | | | | |
| Avicel PH 102 | — | | 327.7 | (29.79%) | — | |
| Prosolve 90 | — | | — | | 331.0 | (30.09%) |
| Disintegrants | | | | | | |
| Sodium Starch | 55 | (5%) | 55 | (5%) | 55 | (5%) |
| Glycolate | | | | | | |
| Glidants | | | | | | |
| Colloidal | 3.3 | (0.3%) | 3.3 | (0.3%) | — | |
| Silicon | | | | | | |
| dioxide | | | | | | |
| Talc | 33 | (3%) | 33 | (3%) | 33 | (3%) |
| Lubricant | | | | | | |
| Magnesium | 11 | (1%) | 11 | (1%) | 11 | (1%) |
| Stearate | | | | | | |
| Total wt per | 1100 | | 1100 | | 1100 | |
| tablet | | | | | | |

Problems with these formulations led to a further investigations employing different type of naproxen sodium having superior physical characterictics together with an effervescent to facilitate the disintegration. Table 2 below shows the formulation

TABLE 2

| | Qty Range (mg)/tablet | |
|---|---|---|
| Ingredients | (% w/w) | |
| Naproxen Sodium (90 micron) | 550 | (50%) |
| Sumatriptan Succinate | 120 | (10.9%) |
| Filler/Binders | | |
| Anhydrous Emcompress and/or | 220-330 | (20-30%) |
| Microcrystalline cellulose | | |
| Disintegrants | | |
| Croscarmellose Sodium and/or | 55 | (5%) |
| Sodium Bicarbonate¹ | 55-110 | (5-10%) |
| Glidant | | |
| Colloidal Silicon Dioxide | 3.3 | (0.3%) |
| Talc | 0-33 | (0-3%) |
| Lubricant | | |
| Magnesium Stearate | 11 | (1%) |

| | |
|---|---|
| Total wt per tablet | 1000-1100 |

¹Sodium bicarbonate, USP or “surface modified” Effersoda by SPI

By combinations of carbonate bases (i.e. sodium bicarbonate) and an acid, an effervescent reaction would take place spontaneously in the presence of moisture to form water and carbon dioxide. Effer-soda™, by SPI Pharma, is a “highly stable, surface modified sodium bicarbonate powder, which has a larger particle size range and in turn better flow characteristics and less sticking propensity”.

However it was found that however superior the physical characteristics of the 90-micron naproxen sodium, the combined direct compression efforts of the two drug substances were unsuccessful.

### EXAMPLE 2

Sumatriptan Succinate Fast Dispersible Tablet (FDT) Granulation and Naproxen Sodium Direct Compression

One of the formulations investigated combining a rapid dispersible formulation of sumatriptan in the combination product was to have rapid disintegration of the sumatriptan succinate material.

The formulation is shown in Table 3.

TABLE 3
Sumatriptan Succinate Granulation and
Naproxen Sodium Direct Compression

| Ingredients | Qty (mg) | Qty/batch (g) |
|---|---|---|
| Naproxen Sodium (90 micron) | 550 | 25 |
| Sumatriptan Succinate | 119 | Xxxxxxx |
| Sumatriptan Succinate:Anhydrous | | 10.8 |
| Emcompress | | |

Filler/Binders

| | | | |
|---|---|---|---|
| Anhydrous Emcompress | 133.1 | (12.1%) | 6.05 |

Disintegrants

| | | | |
|---|---|---|---|
| Croscarmellose Sodium | 110 | (10%) | 5.0 |
| Sodium Bicarbonate | 110 | (10%) | 5.0 |

Glidant

| | | | |
|---|---|---|---|
| Colloidal Silicon dioxide | 3.3 | (0.3%) | 0.15 |

Lubricant

| | | | |
|---|---|---|---|
| Magnesium Stearate | 11 | (1%) | 0.5 |
| Total wt per tablet | 1100 | | 50 |

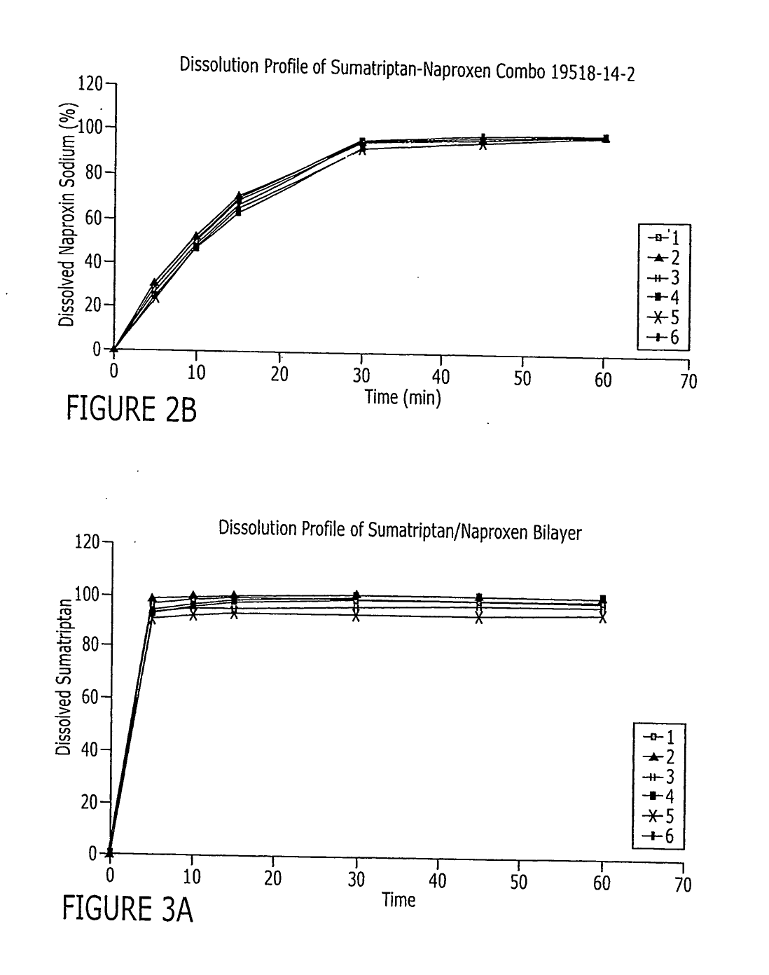

As a result of using the Suma FDT formulation approach, the apparent sticking problems observed with the previous formulations of the formulation was reduced, but not eliminated. FIG. 2 shows a dissolution profile for sumatriptan succinate (left) indicating 33% sumatriptan released in 5 minutes, compared to 95% in 5 minutes for the FDT granulation alone. Naproxen Sodium dissolution (right) is at 67% in 15 minutes, comparable to Anaprox DS.

The dissolution of both components was slower than expected with FDT granulation incorporated into the formulation. This result led to the conclusion that the naproxen sodium was disabling the dissolution of the sumatriptan granules when blended together.

### EXAMPLE 3

The next approach was a bilayer tablet. The Introduction of the FDT granule formulation as a separate layer containing sumatriptan succinate allowed for reduced sticking on the sumatriptan side.

The direct compression processing of the naproxen sodium material included disintegrants, lubricants and fillers A representative formulation composition is shown in Table 4.

TABLE 4
Formulation composition for Bilayer Tablet Containing
Sumatriptan Succinate FDT Granulation and Naproxen
Sodium Direct Compression.

| | Qty | (% |
|---|---|---|
| Ingredients | (mg/tablet) | w/w) |
| Layer 1 | | |
| Sumatriptan Succinate: Anhydrous Emcompress | 238 | 68.0 |
| (in 1:1 ratio) | | |
| Avicel PH 102 (microcrystalline cellulose) | 52.75 | 15.1 |
| Croscarmellose Sodium | 17.5 | 5.0 |
| Sodium Bicarbonate | 40 | 11.4 |
| Magnesium Stearate | 1.75 | 0.5 |
| Total layer weight | 350 | 100 |
| Layer 2 | | |
| Naproxen Sodium (90 micron) | 550 | 68.75 |
| Citric Acid | 56 | 7.0 |
| Croscarmellose Sodium | 24 | 3.0 |
| Effersoda (surface-modified sodium bicarbonate) | 1.20 | 15 |
| Colloidal Silicon dioxide | 2.4 | 0.3 |
| Avicel PH 102 (microcrystalline cellulose) | 35.6 | 4.45 |
| Magnesium Stearate | 12 | 1.5 |
| Total layer weight | 800 | 100 |

Flowability Index = 18.5
Hardness at Compression Force of 15.7 kN = 15 kp

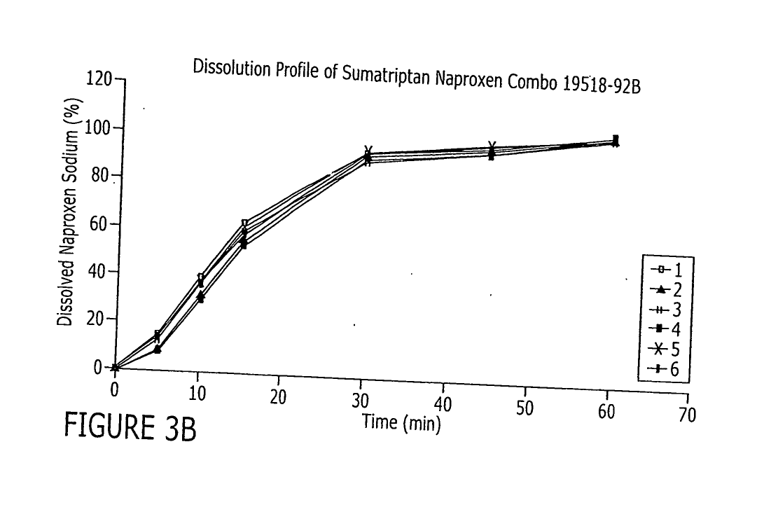

Combining the two formulations as separate layers in a bilayer tablet allowed for the rapid release of sumatriptan succinate from the tablet, without as much interference from naproxen sodium. FIG. 3 shows the dissolution profile for this bilayer approach.

The dissolution profiles for sumatriptan succinate and naproxen sodium from the FDT based bilayer combination tablet shows the sumatriptan succinate was dissolved at 95% in 5 minutes. This result confirmed the benefit of a bilayer tablet in allowing the sumatriptan succinate to release rapidly. Additionally, the Naproxen sodium released is approximately 59% in 15 minutes, very close to the gold standard Anaprox® DS.

### EXAMPLES 4 TO 6

Further bilayer formulations are illustrated in Table 5; these all involved processing of both the sumatriptan succinate FDT layer and the naproxen sodium layer by granulation methods.

TABLE 5

| | | | |
|---|---|---|---|
| | 4 | 5 | 6 |
| Example | Qty | Qty | Qty |
| Ingredients | (mg) | (mg) | (mg) |

Layer 1

| | | | | |
|---|---|---|---|---|
| Intragranular | Sumatriptan | 119 | 119 | 119 |
| | succinate | | | |
| | Anhydrous | 119 | 119 | 119 |
| | Emcompress | | | |
| Extragranular | Avicel | 100 | 100 | 100 |
| | PH 102 | | | |
| | Croscarmel- | 20 | 20 | 20 |
| | lose Sodium | | | |
| | Sodium | 40 | 40 | 40 |
| | Bicarbonate | | | |
| | Magnesium | 2 | 2 | 2 |
| | Stearate | | | |

| | | | |
|---|---|---|---|
| Layer Total | 400 | 400 | 400 |

Layer 2

| | | | | |
|---|---|---|---|---|
| Intragranular | Naproxen | 500 | 500 | 500 |
| | sodium | | | |
| | Avicel | 54 | 54 | 54 |
| | PH 101 | | | |
| | Polyvinyl- | 27 | — | 20.25-33.75 |
| | pyrrolidone, | | | |
| | PVP | | | |
| | Hydroxy- | — | 13.5-33.75 | — |
| | propyl cellu- | | | |
| | lose | | | |
| Extragranular | Aviel | 40 | QS | QS |
| | PH 102 | | | |
| | Croscarmel- | 20.25 | 20.25 | 20.25 |
| | lose Sodium | | | |
| | Colloidal | — | 0-4.73 | 0-4.73 |
| | silicon | | | |
| | dioxide | | | |
| | Talc | 27 | 21.5-53.75 | 21.5-53.75 |
| | Magnesium | 6.75 | 6.75 | 6.75 |
| | Stearate | | | |

| | | | |
|---|---|---|---|
| Layer total | 675 | 675 | 675 |
| TOTAL TABLET | 1075 | 1075 | 1075 |
| CORE WEIGHT | | | |

Preparation of Examples 4 to 6 involved the following standard formulation steps:
Naproxen Sodium layer:
High shear granulation
Fluid bed drying of granulation; milling
Dry blend of granulation and other excipients
Sumatriptan Succinate layer:
High shear granulation
Fluid bed drying of granulation; milling
Dry blend of granulation and other excipients
Compression as a Bilayer Tablet
Conventional aqueous film-coating, e.g. with Opadry, Opaglos

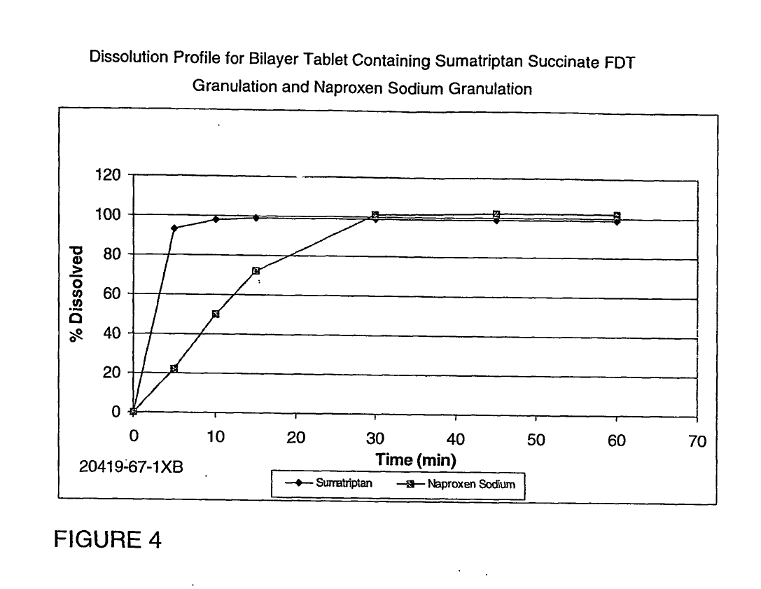

The dissolution profile for the tablet formulation of Example 4 is shown in FIG. 4 (USP I Apparatus, pH 6.8, phosphate buffer, 50 rpm, n=6 tablets). This shows that the sumatriptan succinate was dissolved at 95% in 5 minutes. This result further confirmed the benefit of a bilayer tablet in allowing the sumatriptan succinate to release rapidly. Additionally, the Naproxen sodium released is greater than 70% in 15 minutes, above the gold standard Anaprox® DS.

The application of which this description and claims forms part may be used as a basis for priority in respect of any subsequent application. The claims of such subsequent application may be directed to any novel feature or combination of features described herein. This may take the form of product, composition, process or use claims and may include, by way of example and without limitation, one or more of the following claims.

What is claimed is:

1. A pharmaceutical composition comprising a 5HT₁ receptor agonist or a pharmaceutically acceptable derivative thereof in combination with a NSAID or pharmaceutically acceptable derivative thereof wherein the 5HT₁ receptor agonist and NSAID are located in discrete zones with respect to each other wherein each zone comprises the active ingredient and optionally a carrier.

2. The pharmaceutical composition according to claim 1 comprising a 5HT₁ receptor agonist selected from the group comprising sumatriptan, naratriptan, zolmitriptan, eletriptan, rizatriptan, frovatriptan, almotriptan, avitriptan, donitriptan, alniditan, ALX-0646, LY334370, U1092291, IS159 and PNY142633.

3. The pharmaceutical composition according to claim 2 wherein the 5HT₁ receptor agonist is sumatriptan or naratriptan.

4. The pharmaceutical composition according to claim 3 wherein the 5HT₁ receptor agonist is sumatriptan.

5. The pharmaceutical composition according to claim 4 comprising sumatriptan succinate.

6. The pharmaceutical composition according to claim 1 comprising a NSAID selected from the group consisting of diclofenac, nabumetone, naproxen, ketorolac, ibuprofen, flurbiprofen, ketoprofen, oxaprozin, etodolac, indomethacin, mefanamic acid, tolfenamic acid and COX-2 selective inhibitors such as celecoxib, rofecoxib (VIOXX), valdecoxib, parecoxib, 4-(4-cyclohexyl-2-methyl-5-oxazoly)-2-fluorobenzenesulfonamide (JTE-522), MK633 (etoricoxib), nimesulide, flosulide, DFP, 2-(4-ethoxy-phenyl)-3-(4-methanesulfonyl-phenyl)-pyrazolo[1,5-b]pyridazine, meloxicam, RS57067, piroxicam, NS398, L-745,337 and COX-189.

7. The pharmaceutical composition according to claim 6 wherein the NSAID is COX-189 or naproxen.

8. The pharmaceutical composition according to claim 7 wherein the NSAID is naproxen.

9. The pharmaceutical composition according to claim 8 wherein the naproxen is naproxen sodium.

10. The pharmaceutical composition comprising a 5HT₁ receptor agonist or a pharmaceutically acceptable derivative thereof and a pharmaceutically acceptable carrier in combination with an NSAID, wherein the 5HT₁ receptor agonist and NSAID are each dispersed within its own pharmaceutically acceptable carrier in the pharmaceutical composition.

11. The pharmaceutical composition according to claim 10 wherein the carrier for the 5HT₁ receptor agonist is different in composition to that of the carrier for the NSAID.

12. The pharmaceutical composition according to claim 10 wherein the 5HT₁ receptor agonist and its carrier are substantially in admixture with the NSAID and its carrier.

13. The pharmaceutical composition according to claim 10 in the form of a tablet.

14. The pharmaceutical composition according to claim 1 wherein suitable carriers for the 5HT₁ receptor agonist comprises one or more components selected from: a binding agent, a filler, a lubricant, and effervescent couple, a wicking agent, a glidant, a disintegrant and a wetting agent.

15. The pharmaceutical composition according to claim 1 wherein suitable carriers for the NSAID comprises one or more components selected from: a binding agent, a filler, a lubricant, an effervescent couple, a wicking agent, a glidant, a disintegrant and a wetting agent.

16. The pharmaceutical composition according to claim 1 which is a multilayer tablet wherein the active agents are in separate layers.

17. The pharmaceutical composition according to claim 16 which is a bilayer tablet.

18. The pharmaceutical composition according to claim 1 comprising a 5HT₁ receptor agonist or a pharmaceutically acceptable derivative thereof in combination with an NSAID or pharmaceutically acceptable derivative thereof wherein the 5HT₁ receptor agonist and NSAID are located in discrete zones with respect to each other wherein each zone comprises the active ingredient and receptor agonist is formulated to ensure rapid absorption.

19. The pharmaceutical composition according to claim 18 which is a tablet comprising two or more layers wherein one layer comprising a 5HT₁ receptor agonist or pharmaceutically acceptable derivative thereof is formulated to ensure rapid absorption and another layer comprises a NSAID or pharmaceutically acceptable derivative thereof and optionally a carrier.

20. The pharmaceutical composition according to claim 19 which is a bilayer tablet comprising a layer comprising a 5HT₁ receptor agonist or pharmaceutically acceptable derivative thereof which is formulated to ensure rapid absorption and a layer comprising a NSAID or pharmaceutically acceptable derivative thereof and optionally a carrier.

21. The pharmaceutical composition according to claim 18 wherein the 5HT₁ receptor agonist or pharmaceutically acceptable derivative thereof is formulated together with an effervescent couple in combination with a disintegrant, an insoluble filler and a wicking agent.

22. The pharmaceutical composition according to claim 18 wherein the 5HT₁ receptor agonist or a pharmaceutically acceptable derivative thereof is formulated together with the base component of an effervescent couple, a disintegrant, and an insoluble filler, wherein the base component comprises from about 5 to about 50% by weight, the disintegrant comprises from about 0.5 to about 10% by weight, and the insoluble filler comprises from about 20 to about 99% by weight, said insoluble filler including a wicking agent which comprises from about 1 to about 99% by weight, based on the dry weight of the layer of the dosage form, wherein greater than about 70% of the active ingredient is dissolved in simulated gastric fluid (SGF) within five minutes in USPII apparatus at the discriminating paddle speed of 10 rpm.

23. The pharmaceutical composition according to claim 1 wherein the 5HT₁ receptor agonist layer comprises a 5HT₁ receptor agonist or a pharmaceutically acceptable derivative thereof which comprises from about 0.001 to about 55% by weight, the base component of the effervescent couple comprises from about 5 to about 50% by weight, the disintegrant comprises from about 0.5 to about 10% by weight, the insoluble filler, including the wicking agent, comprises from about 35 to about 80% by weight, and the wicking agent comprises from about 1 to about 80% by weight, based on the dry weight of the layer of the dosage form.

24. The pharmaceutical composition according to claim 18 wherein in the NSAID layer the NSAID or pharmaceutically acceptable derivative thereof is formulated for rapid absorption.

25. The pharmaceutical composition according to claim 18 wherein in the NSAID layer the NSAID or pharmaceutically acceptable derivative thereof is formulated together with an effervescent couple in combination with a disintegrant, an insoluble filler, and a wicking agent.

26. The pharmaceutical composition according to claim 18 wherein the NSAID or a pharmaceutically acceptable derivative thereof, is formulated together with the base component of an effervescent couple, a disintegrant, and an insoluble filler, wherein the base component comprises from about 5 to about 50% by weight, the disintegrant comprises from about 0.5 to about 20% by weight, and the insoluble filler comprises from about 30 to about 80% by weight, said insoluble filler including a wicking agent which comprises from about 1 to about 60% by weight, based on the dry weight of the layer of the dosage form, wherein greater than about 25% of the active ingredient is dissolved in simulated intestinal fluid within five minutes in USPII apparatus at the discriminating paddle speed of 30 rpm.

27. The pharmaceutical composition according to claim 1, wherein the NSAID layer comprises a NSAID or a pharmaceutically acceptable derivative thereof which comprises from about 1 to about 90% by weight, the base component of the effervescent couple comprises from about 5 to about 50% by weight, the disintegrant comprises from about 0.05 to about 10% by weight, the insoluble filler, including the wicking agent, comprises from about 10 to about 99% by weight based on the dry weight of the layer of the dosage form.

28. The pharmaceutical composition according to claim 1 which is a bilayer tablet wherein the 5HT₁ receptor agonist layer comprises a 5HT₁ receptor agonist or a pharmaceutically acceptable derivative thereof which comprises from about 0.001 to about 55% by weight, the base component of the effervescent couple comprises from about 5 to about 50% by weight, the disintegrant comprises from about 0.5 to about 10% by weight, the insoluble filler, including the wicking agent, comprises from about 35 to about 80% by weight, and the wicking agent comprises from about 1 to about 80% by weight, based on the dry weight of the layer of the dosage form and the NSAID layer comprises a NSAID or a pharmaceutically acceptable derivative thereof which comprises from about 1 to about 90% by weight, the base component of the effervescent couple comprises from about 5 to about 50% by weight, the disintegrant comprises from about 0.05 to about 10% by weight, the insoluble filler, including the wicking agent, comprises from about 10 to about 99% by weight, based on the dry weight of the layer of the dosage form.

29. The pharmaceutical composition according to claim 1 wherein the 5HT₁ receptor agonist zone comprises a 5HT₁ receptor agonist or a pharmaceutically acceptable derivative thereof which comprises sumatriptan or naratriptan or a pharmaceutically acceptable derivative thereof, the base component of the effervescent couple comprises sodium bicarbonate, the disintegrant comprises croscarmellose sodium, and the insoluble filler comprises microcrystalline cellulose.

30. The pharmaceutical composition according to claim 1, wherein the 5HT₁ receptor agonist zone comprises a 5HT₁ receptor agonist or a pharmaceutically acceptable derivative thereof which comprises sumatriptan or naratriptan or a pharmaceutically acceptable derivative thereof, the base component of the effervescent couple comprises sodium bicarbonate, the disintegrant comprises croscarmellose sodium, and the insoluble filler comprises dibasic calcium phosphate, preferably anhydrous dibasic calcium phosphate.

31. The pharmaceutical composition according to claim 1, wherein the 5HT₁ receptor agonist zone comprises a 5HT₁ receptor agonist or a pharmaceutically acceptable derivative thereof which comprises sumatriptan or a pharmaceutically acceptable derivative thereof the base component of the effervescent couple comprises sodium bicarbonate, the disintegrant comprises croscarmellose sodium, and the insoluble filler comprises anhydrous dibasic calcium phosphate or microcrystalline cellulose or a mixture thereof.

32. The pharmaceutical composition according to claim 20 comprising 85 mg sumatriptan in the form of 119 mg sumatriptan succinate and 550 mg naproxen sodium equivalent to 500 mg naproxen.

33. The pharmaceutical composition according to claim 20 comprising 85 mg sumatriptan in the form of 119 mg sumatriptan succinate and 500 mg naproxen sodium equivalent to 454.5 mg naproxen.

34. The pharmaceutical composition according to claim 1, for use in the treatment of conditions associated with cephalic pain selected from the group consisting of cluster headache, chronic paroxysmal hemicrania, headache associated with vascular disorders, headache associated with substances or their withdrawal, rebound headache, tension headache, and migraine.

35. A method of treating a mammal suffering from or susceptible to conditions associated with cephalic pain selected from the group consisting of cluster headache, chronic paroxysmal hemicrania, headache associated with vascular disorders, headache associated with substances or their withdrawal, rebound headache, tension headache, and migraine, which comprises oral administration of a solid-dosage form pharmaceutical composition according to claim 1.

36. The method of claim 36 wherein the mammal is a human.

37. The method of claim 36 wherein the condition is migraine.
